SEQUENCE LISTING (8 sequences)

SEQ ID NO: 1 - DNA, 465 bp, Artificial sequence
  human fibroblast growth factor 2
atggcagccgggagcatcaccacgctgcccgccttgcccgaggatggcggcagcggcgcttcccgcccggccacttcaaggaccccaagcggctgtactgcaaaaacgggggcttcttctgcgcatccaccccgacggccgagttgacggggtccgggagaagagcgaccctcacataagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 2 - protein, 155 aa, Artificial sequence [sequence omitted]

SEQ ID NO: 3 - DNA, 1167 bp, Artificial sequence
  maltose-binding protein
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttca

SEQ ID NO: 4 - protein, 389 aa, Artificial sequence
  maltose-binding protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRIS

SEQ ID NO: 5 - DNA, 1654 bp, Artificial Sequence
  MBP-FGF recombinant protein
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccccgccttgcccgaggatggcagccgggagcatcaccacgctgcccgccttgcccgaggatggcggcagcggcgcttcccgcccggccacttcaaggaccccaagcggctgtactgcaaaaacgggggcttcttctgcgcatccaccccgacggccgagttgacggggtccgggagaagagcgaccctcacataagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 6 - protein, 543 aa, Artificial Sequence
  MBP-FGF recombinant protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 7 - DNA, 29 bp, Artificial Sequence
  bFGF-F forward primer
ccgaattccccgccttgcccgaggatggc

SEQ ID NO: 8 - DNA, 32 bp, Artificial Sequence
  FGF2-R reverse primer
caaagctttcagctcttagcagacattggaag

(19) United States
(12) Patent Application Publication
(10) Pub. No.: US 2012/0122156 A1
(43) Pub. Date: May 17, 2012

(54) RECOMBINANT PROTEIN OF FIBROBLAST GROWTH FACTOR HAVING ADHESIVE ACTIVITY FOR STEM CELLS AND METHOD FOR CULTURING STEM CELLS USING THE SAME

(76) Inventors: Sang-Heon Kim, Seoul (KR); Soo Hyun Kim, Seoul (KR); Young Mee Jung, Seoul (KR); Min Han, Seoul (KR)

(21) Appl. No.: 13/320,193

(22) PCT Filed: Oct. 14, 2009
(86) PCT No.: PCT/KR2009/005899

§ 371 Date: Nov. 11, 2011

(30) Foreign Application Priority Data
May 13, 2009 (KR) 10-2009-0041846

(51) Int. Cl. C12N 5/071; C12P 21/02; C12N 15/63; C12N 1/21; C07K 19/00; C12N 15/62

(52) U.S. Cl. 435/69.7; 435/405; 530/399; 536/23.4; 435/320.1; 435/252.3; 435/252.33

(57) ABSTRACT

The present invention relates to a recombinant protein of a fibroblast growth factor (FGF) having an adhesive activity for stem cells and a method for culturing stem cells using the same. More particularly, the present invention relates to a recombinant protein having an adhesive activity for stem cells by fusion of a polypeptide linker at amino terminal of FGF, and a method for culturing stem cells using immobilized FGF comprising: fixing the recombinant protein in a culture vessel with a hydrophobic surface using amino terminal of the polypeptide linker, adhering stem cells on the recombinant protein-fixed culture vessel, and culturing the stem cells.

### TECHNICAL FIELD

The present invention relates to a recombinant protein capable of adhering to stem cells, in which a polypeptide linker is fused to the amino terminus of fibroblast growth factor (FGF), and to a method of culturing stem cells using immobilized fibroblast growth factor, the method comprising immobilizing the recombinant protein on a culture plate having a hydrophobic surface and then allowing stem cells to bind to the surface and culturing the stem cells.

### BACKGROUND ART

Cell adhesion to the surface of biomaterials occurs by various mechanisms and can be classified into specific cell adhesion mediated by biological recognition and non-specific adhesion governed by electrostatic or surface energy. Specific cell adhesion occurs when specific peptide ligands present in extracellular matrix (ECM) proteins (e.g., collagen, fibronectin, laminin, etc.), such as Arg-Gly-Asp (RGD), bind to integrins that are adhesion receptors present on the cell membrane. On the other hand, non-specific cell adhesion is a process by which the surface to be adhered by cells is made electropositive to induce the adhesion of the cells since cell membranes mainly composed of phospholipids are electrically negative. Most currently available tissue cell culture plates have surfaces which are made electropositive by plasma treatment based on such non-specific cell adhesion principle. In addition, cell adhesion can be induced if the surface to be adhered by cells is imparted with surface energy corresponding to that of the cell membrane.

It is known that good cell adhesion occurs at about 60°, the contact angle of water, even though there is a slight difference between cells.

In the prior art, most cells, including stem cells, were cultured in a culture plate either coated with ECM or having an electrically positive surface as described above. Recently, artificial cell adhesion ligands genetically engineered from receptor ligands targeting various receptors on the cell surface were developed. For example, it was reported that, when epithelial tumor cells or embryonic stem cells were cultured on a soluble EGF or gelatin-adsorbed surface in a polystyrene culture plate immobilized with epidermal growth factor (EGF)-Fc or cadherin-Fc, the cells showed different biochemical and cell-biological properties (Ogiwara K. et al., Biotech. Letters 27: 1633-1637, 2005; Nagaoka M. et al, PLoS ONE 1: e15, 2006).

However, when Fc is used as described above, the carboxyl terminus of Fc is required for physical adsorption to a hydrophobic surface, and the amino terminus of Fc is required for binding to a physiologically active target polypeptide. For this reason, the use of Fc is limited if the carboxyl terminus of a physiologically active polypeptide is essential for activity. In other words, immobilization of Fc is impossible in the case of a physiologically active polypeptide, such as fibroblast growth factor (FGF), the carboxyl terminus of which plays an important role in maintaining activity.

FGF is essential for the maintenance of homeostasis in vivo, such as the restoration of living tissue or a response to a wound, and is known to regulate the proliferation, migration, differentiation and survival of cell in the embryonic stage. Also, FGF is very important for the induction of differentiation or proliferation in culture of adipose-derived stem cells, mesenchymal stem cells, embryonic and the like, and it exhibits biological functions by binding to FGF receptor (FGFR) and heparin- or heparan-sulfate proteoglycan (HSPG). A study on the culture of human umbilical vein endothelial cells (HUVECs) on a surface immobilized with a recombinant protein obtained by binding FGF to a cocoon-derived protein was reported (Hino R et al., Biomaterials 27: 5715-5724, 2006). However, in the above study, there is no mention of cell adhesion, and it is not easy to obtain the cocoon-derived protein. Thus, the cocoon-derived protein is difficult to use in the mass culture of specific cells.

Accordingly, the present inventors have made extensive efforts to develop an efficient method for culturing stem cells using FGF and, as a result, discovered that stem cells can be efficiently cultured by preparing a recombinant protein in which the carboxyl terminus of a polypeptide linker, which can be expressed and purified in the form of a recombinant protein, is fused to the amino terminus of FGF, immobilizing the recombinant protein on a culture plate having a hydrophobic surface by simple physical adsorption via a hydrophobic domain located at the amino terminus of the linker, and then allowing stem cells to bind to the immobilized recombinant protein while the FGF portion of the recombinant protein maintains its original physical activity, thereby completing the present invention.

### Disclosure

### Technical Problem

Therefore, it is an object of the present invention to provide a method of efficiently culturing stem cells using fibroblast growth factor immobilized on a hydrophobic surface, in which fibroblast growth factor is fused to a polypeptide linker so as to form a recombinant protein capable of adhering to stem cells.

### Technical Solution

To achieve the above object, the present invention provides a polypeptide linker-FGF recombinant protein capable of adhering to stem cells, in which the amino terminus of fibroblast growth factor is fused to the carboxyl terminus of a polypeptide linker.

The present invention also provides a polynucleotide encoding said recombinant protein.

The present invention also provides an expression vector comprising said nucleotide, and bacteria transformed with the expression vector.

The present invention also provides a method for preparing said recombinant protein, the method comprising culturing said transformed bacteria.

The present invention also provides a method for culturing stem cells using immobilized fibroblast growth factor (FGF), the method comprising the steps of:

1) immobilizing a recombinant protein, comprising a polypeptide linker fused to the amino terminus of fibroblast growth factor (FGF), on a culture plate having a hydrophobic surface, using the amino terminus; and

2) allowing stem cells to adhere to the recombinant protein-immobilized culture plate and culturing the cells.

### Advantageous Effects

According to the present invention, FGF binding to FGF receptor and HSPG, which are expressed on the membrane of stem cells, can be immobilized on a hydrophobic surface by simple physical adsorption using a polypeptide linker, which is expressed in large amount and is easy to purify, while the physiological activity of FGF intact. The surface immobilized with FGF can induce the adhesion of stem cells thereto by interaction between FGF and the stem cells, and particularly, can control the morphology of the adhered stem cells by integrin-independent cell adhesion. This control of the morphology of stem cells makes it possible to effectively induce the differentiation of the stem cells. Thus, the present invention can be applied to culture technology related to the differentiation and proliferation of stem cells.

### BRIEF DESCRIPTION OF THE DRAWINGS

The foregoing and other objects, features and advantages of the present invention will become more apparent from the following detailed description when taken in conjunction with the accompanying drawings in which:

FIG. 1 shows the results of SDS-PAGE analysis of a recombinant protein consisting of a maltose-binding protein (MBP) and fibroblast growth factor 2 (FGF2) according to the present invention;

FIG. 2 is a set of graphs showing the proliferation rate of HUVEC cells, treated with the MBP-FGF recombinant protein, as a function of the concentration of the recombinant protein;

FIG. 3 is a set of photographs showing the results of observing HUVEC cells with a phase contrast microscope after culturing the cells for 3 days under the conditions of treatment with the MBP-FGF recombinant protein according to the present invention (C), treatment with FGF2 alone (B), and non-treatment (A),

FIG. 4 is a graph showing the rate of adhesion of adipose-derived stem cells to a polystyrene surface, immobilized with the MBP-FGF recombinant protein according to the present invention, as a function of the concentration of the recombinant protein;

FIG. 5 is a set of graphs showing a comparison between the rate of adhesion of adipose-derived stem cells to a polystyrene surface, immobilized with the MBP-FGF recombinant protein according to the present invention, and the rates of adhesion to various different surfaces;

FIG. 6 is a set of phase-contrast micrographs showing the results of observing a change in the morphology of adipose-derived stem cells, cultured in a serum-free medium on a polystyrene surface immobilized with the MBP-FGF recombinant protein;

FIG. 7 is a set of graphs showing the rate of adhesion of mesenchymal stem cells to a polystyrene surface, immobilized with the MBP-FGF according to the present invention, as a function of the concentration of the recombinant protein.

### BEST MODE

### Mode for Invention

Hereinafter, exemplary embodiments of the present invention will be described in detail.

The present invention provides a method of using fibroblast growth factor immobilized on the surface of a culture plate to efficiently culture stem cells.

For this purpose, the present invention uses a polypeptide linker, which can be recombinantly expressed in large amount and can be easily purified, to provide a polypeptide linker-FGF recombinant protein capable of adhering to stem cells, in which the amino terminus of fibroblast growth gactor (FGF) is fused to the carboxyl terminus of the polypeptide, as well as a polynucleotide encoding the recombinant protein.

The present invention is characterized in that FGF in a recombinant protein form essential for the differentiation and proliferation of stem cells is immobilized on a hydrophobic surface using a polypeptide linker while maintaining the original biological activity, and then stem cells are allowed to the surface using the activity of adhesion of the immobilized FGF to the stem cells, thereby inducing efficient culture of the stem cells.

FGF is a growth factor that binds to the FGF receptor or HSPG present in the cell membrane to play important biological functions in the differentiation or proliferation of adipose-derived stem cells, mesenchymal stem cells, embryonic stem cells or the like during culture of these stem cells. It has a nucleotide sequence set forth in SEQ ID NO: 1 and an amino acid sequence set forth in SEQ ID NO: 2.

The polypeptide that is used as a linker in the present invention binds to the amino terminus of FGF via its carboxyl terminus and adsorbs onto a culture plate having a hydrophobic surface via a hydrophobic domain present at its amino terminus. Any polypeptide may be used as a polypeptide linker in the present invention, ao long as it can be recombinantly expressed in large amount, can be easily purified and does not affect the culture of stem cells. Examples of this polypeptide linker include maltose-binding protein (MBP), hydrophobin, hydrophobic cell penetrating peptides (CPPs) and the like.

In one preferred embodiment of the present invention, the polypeptide linker is, for example, maltose-binding protein (MBP). MBP is a periplasm protein of Escherichia coli, which is involved in the transport of sugars such as maltose or maltodextrin. MBP has a nucleotide sequence set forth in SEQ ID NO: 3 and an amino acid sequence set forth in SEQ ID NO: 4.

MBP is mainly used to produce useful foreign protein in the form of recombinant proteins and is encoded by the gene malE. If a foreign protein gene is inserted downstream of the cloned malE gene and expressed in cells, a recombinant protein consisting of two fused proteins can be easily produced in large amount. Particularly, if a protein to be expressed has a small size or if a foreign protein has reduced stability in host cells, it is advantageously expressed in the form of a recombinant protein in cells using MBP. A foreign protein expressed from a gene bound to the malE gene can be separated from the gene using the affinity of binding of MBP to maltose. For example, the desired protein can be simply eluted by allowing a cell lysis to react with an amylase (maltose polymer)-coated resin, washing the reacted resin several times to remove other contaminated proteins, and then adding high-concentration maltose thereto so as to bind to MBP. Because the use of MBP enables the desired protein to be very easily separated and purified after the desired protein had been expressed in cells, systems for expressing recombinant proteins comprising MBP are frequently used to produce target foreign proteins worldwide.

In one embodiment of the present invention, based on the fact that maltose-binding protein (MBP) has an advantage in that it is easily expressed and purified because of its high ability to bind to maltose, a protein that is expressed in E. coli, the present invention provides a method comprising preparing a recombinant protein in which the carboxyl terminus of maltose is fused with the amino end of FGF, immobilizing the recombinant protein on the hydrophobic surface of a culture plate by simple physical adsorption using the hydrophobic domain of MBP as a linker, allowing stem cells to adhere to the surface and culturing the stem cells while still maintaining the biological activity of the FGF moiety of the immobilized recombinant protein. In this embodiment, the carboxyl terminus of MBP is used to bind to FGF so as to prepare the recombinant protein, whereas the amino terminus comprising the hydrophobic domain is used for physical adsorption to the hydrophobic surface in the next step.

The sequencing of the recombinant protein revealed that the amino terminus of fibroblast growth factor (FGF) is fused to the carboxyl terminus of maltose-binding protein, and the MBP-FGF recombinant protein capable of adhering to stem cells has an amino acid sequence of SEQ ID NO: 6 which is encoded by a polynucleotide having a nucleotide sequence of SEQ ID NO: 5.

Furthermore, the present invention provides a recombinant expression vector comprising a polynucleotide encoding the polypeptide linker-FGF recombinant protein, and bacteria transformed with the recombinant vector.

The term “expression vector”, as used herein, which is a vector capable of expressing a target protein in a suitable host cell, refers to a genetic construct that comprises essential regulatory elements to which a gene insert is operably linked thereto in such a manner as to be expressed in a host cell.

The term “operably linked”, as used herein, refers to a functional linkage between a nucleic acid expression control sequence and a second nucleic acid sequence coding for a target protein in a manner that allows general functions. For example, when a nucleic acid sequence coding for a protein is operably linked to a promoter, the promoter may affect the expression of a coding sequence. The operable linkage to a recombinant vector may be prepared using a genetic recombinant technique well known in the art, and site-specific DNA cleavage and ligation may be carried out using enzymes generally known in the art.

The expression vectors useful in the present invention include, but are not limited to, plasmid vectors, cosmid vectors and viral vectors. A suitable expression vector includes expression regulatory elements, such as a promoter, an operator, an initiation codon, a termination codon, a polyadenylation signal and an enhancer, and a signal sequence or leader sequence for membrane targeting or secretion, and may be prepared in various constructs according to the intended use. The promoter of the vector may be constitutive or inducible. Also, the expression vector includes a selectable marker for selecting a host cell containing a vector, and, in the case of being replicable, includes a replication origin.

In a preferred embodiment of the present invention, an expression vector comprising a recombinant gene fragment in which a FGF gene is linked to the carboxyl terminus of MBP is prepared by amplifying FGF by polymerase chain reaction (PCR) and cloning the amplified FGF gene into a vector containing the MBP gene. The inventive recombinant expression vector prepared as such may be, for example, pMAL-c2X-FGF2. The recombinant expression vector pMAL-c2X-FGF2 means a vector in which the FGF gene amplified by PCR is inserted into the open reading frame (ORF) of the carboxyl terminus of the MBP gene in a pMAL-c2X vector (New England Biolabs).

Host cells are transformed with the recombinant expression vector prepared as described above to obtain a transformed strain. Since expression level and modification of proteins vary depending on host cells, host cells that are most suitable for purposes should be selected and used. Host cells include, but not limited to, prokaryotic host cells such as Escherichia coli, Bacillus subtilis, Streptomyces sp., Pseudomonas sp., Proteus mirabilis, or Staphylococcus sp. In addition, lower eukaryotic cells such as fungi (e.g., Aspergillus sp.), yeasts (e.g., Pichia pastoris, Saccharomyces cerevisiae, Schizosaccharomyces, Neurospora crassa, etc.), insect cells, plant cells, or cells derived from higher eukaryotes including mammals may be used as host cells.

In the present invention, the host cell may preferably be E. coli. After E. coli was transformed with the recombinant vector of the present invention, a large amount of the polypeptide linker-FGF recombinant protein can be expressed from the transformed E. coli. Transformation may be carried out via methods being able to introduce nucleic acids into host cells and may be performed by any transformation techniques well known in the art. Preferably, the methods include, but are not limited to, microprojectile bombardment, electroporation calcium, phosphate (CaPO₄) precipitation, calcium chloride (CaC₁₂) precipitation, PEG-mediated fusion, microinjection, and liposome-mediated method.

In a preferred embodiment of the present invention, the recombinant expression vector pMAL-c2X-FGF2 comprising a recombinant gene fragment in which FGF is fused to the carboxyl terminus of MBP was transformed into E. coli K12 TB1 to prepare transformed bacteria expressing the MBP-FGF recombinant protein. The transformed bacterial strain was deposited with Korean Collection for Type Cultures (KCTC), Korea Institute of Bioscience and Biotechnology, on Apr. 28, 2009 under accession number KCTC-11505BP.

The present invention provides a method for producing the polypeptide linker-FGF recombinant protein, comprising culturing the transformed bacteria under suitable conditions, and then collecting the recombinant protein from the culture.

The production method is carried out by culturing the bacteria, transformed with the recombinant expression vector, in suitable media and conditions, such that a polynucleotide encoding the polynucleotide linker-FGF recombinant protein is expressed in the bacterial cells. Methods of expressing the recombinant protein by culturing the transformant are well known in the art. For example, it may be carried out by inoculating a transformant in a suitable medium, performing a subculture, transferring the same to a main culture medium, culturing it under suitable conditions, for example, in the presence of the gene expression inducer, isopropyl-β-D-thiogalactoside (IPTG), thereby, inducing the expression of the recombinant protein. Typically, a medium used in the culturing should contain all nutrients essential for the growth and survival of cells. The medium should contain a variety of carbon sources, nitrogen sources and trace elements. After the culture has been completed, it is possible to collect a “substantially pure” recombinant protein from the culture. The term “substantially pure” means that the recombinant protein of the preset invention and polynucleotide encoding the same are essentially free of other proteins derived from the host cells.

The recombinant protein expressed in the transformed bacteria may be recovered by various isolation and purification methods known in the art. Conventionally, cell lysates are centrifuged to remove cell debris and impurities, and then subjected to precipitation, e.g. salting out (ammonium sulfate precipitation and sodium phosphate precipitation), solvent precipitation (protein fragment precipitation using acetone, ethanol, etc.). Further, dialysis, electrophoresis and various column chromatographies may be performed. With respect to the chromatography, ion exchange chromatography, gel permeation chromatography, HPLC, reverse phase HPLC, affinity chromatography, and ultrafiltration may be used alone or in combination (Maniatis et al., Molecular Cloning: A Laboratory Manual, Cold Spring Harbor Laboratory, Cold Spring Harbor, N.Y., 1982; Sambrook et al., Molecular Cloning: A Laboratory Manual, 2d Ed., Cold Spring Harbor Laboratory Press, 1989; Deutscher, M., Guide to Protein Purification Methods Enzymology vol. 182. Academic Press. Inc., San Diego, Calif., 1990).

In a preferred embodiment of the present invention, the MBP-FGF recombinant protein is separated and purified by affinity column chromatography using a substance capable of binding to maltose, for example, amylase. FGF expressed in the form of a recombinant protein with MBP by the above procedure binds to MBP via its amino terminus, and the carboxyl terminus playing an important role in maintaining the activity of FGF can maintain its original biological activity, because it is exposed to the outside.

As used herein, the term “maintaining biological activity” means that FGF fused to the polypeptide linker maintains 50% or more, preferably 60% or more, and more preferably 70% or more of its original biological activity or function. Even more preferably, the fused FGF maintains 80% or more, and most preferably 90% or more of its original biological activity or function. In the most preferable embodiment of the present invention, FGF, expressed and purified in the form of a recombinant protein with the polypeptide linker, maintains 99% or more of its original biological activity or function.

Thus, the polypeptide linker-FGF recombinant protein according to the present invention can spontaneously physically adsorb onto a hydrophobic surface due to MBP, and can induce the adhesion of stem cells due to FGF. Thus, it is useful not only for studies on the differentiation and proliferation of stem cells, but also in the regenerative medical fields, such as cell therapy and tissue engineering. The present invention provides a method of culturing stem cells using the polypeptide linker-FGF recombinant protein obtained as described above.

Specifically, the method for culturing stem cells according to the present invention comprises the steps of:

1) immobilizing a recombinant protein, in which a polypeptide linker is fused to the amino terminus of fibroblast growth factor (FGF), on a culture plate having a hydrophobic surface using the amino terminus of the polypeptide linker, thus preparing a bioactive surface; and

2) allowing stem cells to bind to the recombinant protein-immobilized culture plate and culturing the stem cells.

Step 1) is a step of providing the polypeptide linker-FGF fusion protein capable of adhering to stem cells, in which the amino terminus of FGF is fused to the carboxyl terminus of the polypeptide linker. The recombinant protein can be prepared using a conventional chemical synthesis or genetic recombination technique known in the art.

In one preferred embodiment of the present invention, the MBP-FGF recombinant protein capable of adhering to stem cells is used in which the amino terminus of fibroblast growth factor (FGF) is fused to the carboxyl terminus of maltose-binding protein (MBP).

Step 2) is a step of immobilizing the recombinant protein, obtained in step 1), on a culture plate having a hydrophobic surface, thereby preparing a bioactive surface having an excellent activity of binding to stem cells. The immobilization does not require does not special treatment and is spontaneously achieved by physical adsorption to the hydrophobic surface using a hydrophobic domain located at the amino terminus of the linker in the recombinant protein.

As used herein, the term “bioactive surface” refers to a surface that can interact directly with stem cells, because FGF is immobilized on a hydrophobic surface in the form of a recombinant protein using a polypeptide linker while maintaining its original biological activity so that it is exposed to the outside. In other words, according to the above immobilization method, when FGF required for the culture of stem cells is immobilized on the hydrophobic surface of a cell culture plate using the polypeptide linker and stem cells are then cultured on the culture plate, FGF and the stem cells can interact directly with each other so that the stem cells adhere to the FGF, thus promoting the culture of the stem cells.

Specifically, in one preferred embodiment of the present invention, the MBP-FGF recombinant protein is diluted in a suitable buffer, for example, buffered phosphate saline (PBS), Tween 20/PBS, Tris-HCl buffer, or bicarbonate buffer, at a concentration of 1 ng/mL to 0.5 mg/mL, after which the dilution is added and allowed to react with a hydrophobic surface at a temperature of 4˜25° C. for 1 to 24 hours. Then, the hydrophobic domain located at the amino terminus of MBP physically adsorbs onto the hydrophobic surface so that the recombinant protein is immobilized on the hydrophobic surface. Herein, the concentration of the MBP-FGF recombinant protein immobilized on the hydrophobic surface is preferably 5 to 100 μg/mL.

Examples of a hydrophobic surface suitable for use in the present invention include a silanized surface, a carbon nanotube (CNT) surface, a hydrocarbon-coated surface, a polymer (e.g., polystyrene, polycarbonate, polypropylene, polyethylene, Teflon, polytetrafluoroethylene or polyester containing biodegradable polymer, etc.) surface, a metal (e.g., stainless steel, titanium, gold, platinum, etc.) surface and the like.

Step 2) is a step of allowing stem cells to adhere to the bioactive surface immobilized with the polypeptide linker-FGF recombinant protein in step 1) and culturing the stem cells. The stem cells useful in step 2) include cells that remain undifferentiated while retaining the capability to differentiate into all types of cells constructing the body, such as blood vessels, neurons, blood, cartilage, etc., in particular, multipotent adult stem cells that are activated only in tissues having the same characteristics as their original tissue. Examples of such stem cells include adipose-derived stem cells, mesenchymal stem cells, bone marrow stem cells, umbilical cord blood stem cells, neural stem cells, etc.

The recombinant protein immobilized on the bioactive surface easily bind to the FGF receptor or HSPG present in the membrane of stem cells, because FGF important for cell recognition is exposed to the outside. Thus, the recombinant protein can play an important role in regulating the function of cells. Thus, when FGF required for the culture of stem cells is immobilized on the hydrophobic surface of a cell culture plate using the polypeptide linker and then stem cells are cultured in the culture plate, the culture of the stem cells can be promoted by the direct interaction between the stem cells FGF. Herein, when the polypeptide linker-FGF recombinant protein according to the present invention together with an extracellular matrix (ECM) such as collagen, fibronectin or laminin is immobilized on the hydrophobic surface, the adhesion thereof to stem cells can be improved.

Hereinafter, the present invention will be described in further detail with reference to examples. It is to be understood, however, that these examples are for illustrative purposes only and are not intended to limit the scope of the present invention.

### REFERENCE EXAMPLE 1

### Cleavage of DNA Using Restriction Enzyme and Collection of Fragment

The restriction enzymes and buffer used in this Example were purchased from Enzynomics. A reaction was carried out in a sterilized 1.5 mL eppendorf tube with a reaction volume of 20-30 μL at 37° C. for 4-5 hours. The composition of 10× buffer used for the restriction enzyme reaction was as follows:
1) 10× Enzynomics buffer Ez buffer: 100 mM Tris-HCl (pH 7.5, 25° C.), 50 mM NaCl, 10 mM MgCl₂, 0.025% Triton X-100, and
2) 10× Enzynomics buffer Ez buffer: 10 mM Tris-HCl (pH 7.5, 25° C.), 50 mM NaCl, 10 mM MgCl₂, 1 mM dithiothreitol.

To recover DNA fragments, electrophoresed agarose gel was irradiated by a UV transilluminator (Avegene) and gels containing the DNA fragments were collected by cutting. Then, the fragments were isolated using a gel extraction kit (Qiagene).

### REFERENCE EXAMPLE 2

### Treatment with Bacterial Alkaline Phosphatase

BAP solution used in treatment with bacterial alkaline phosphatase (BAP) was purchased from Fermentas Co. A reaction was carried out in a sterilized 1.5 mL eppendorf tube with a reaction volume of 50 μL at 60˜65° C. for 1 hour. 1 M Tris-HCl buffer (pH 8.0; Bioneer) was used for the BAP reaction.

### REFERENCE EXAMPLE 3

### Ligation Reaction

A ligation reaction was performed using a DNA ligation kit (DNA Ligation Kit Ver 2.1, Takara), after a vector and an insert were mixed at the ratio of 1:3 and the reaction volume was adjusted to 10-20 μL. The reaction was carried out at 16° C. at least for 12 hours.

### REFERENCE EXAMPLE 4

### Transformation of E. coli

E. coli K12 TB1 (New England Biolabs) was used as a host cell for transformation. The cells were inoculated in 60 mL of liquid medium (10 g/L bacto-tryptone, 5 g/L yeast extract, 10 g/L NaCl), followed by shaking-culture at 37° C. until OD₆₀₀ reached 0.4-0.6. The cultured cells were dispensed into a 1.5 ml eppendorf tube, followed by centrifugation to harvest the cells. 300 μL of 50 mM CaCl₂ was added to the harvested cells, followed by mild vortexing. To harvest the cells, centrifugation was performed again. 300 μL of 50 mM CaCl₂ was added to the harvested cells to uniformly disperse the cells, which were then allowed to stand at 0° C. for 30 minutes. The cell solution was centrifuged and the supernatant was discarded. The remaining cells were uniformly dispersed in 150 μL of a cold solution comprising 50 mM CaCl₂ and 15% glycerol. The cell suspension was stored in a freezer.

### REFERENCE EXAMPLE 5

### Synthesis of Oligonucleotide

A primer pair used in a polymerase chain reaction (PCR) for amplifying a gene encoding Target FGF was synthesized using oligonucleotide synthesis service (Bioneer).

### REFERENCE EXAMPLE 6

### Polymerase Chain Reaction

50 ng of a template and 10 μM of each of forward primer and reverse primer were mixed with distilled water to make a total volume of 10 μL, and then hot start PCR premix (Bioneer) was added thereto. The reaction mixture was subjected to PCR using a T-gradient thermo block (Applied Biometra) under the following conditions: denaturation at 95° C. for 1 min; and then 31 cycles of 30 sec at 94° C., 30 sec at 55° C., 1 min 68° C.; followed by final extension at 72° C. for 5 min. The amplified product was purified with a PCR purification kit (Bioneer) and electrophoresed on agarose gel. The agarose gel was irradiated by a UV transilluminator (Avegene) to cut the desired fragment. The amplified DNA was isolated from the recovered gel fragment using a gel extraction kit (Qiagene).

### REFERENCE EXAMPLE 7

### Isolation of Multipotent Stem Cells From Adipose Tissue

Normal human subcutaneous adipose tissue obtained from the Department of Plastic Surgery, the Catholic University of Korea, was washed three times with 2% penicillin/streptomycin containing PBS to remove contaminated blood, and then was finely chopped with surgical scissors. The adipose tissue was immersed in tissue lysis buffer (serum-free DMEM+1% BSA(w/v)+0.3% collagenase type 1) and stirred for 2 hours at 37° C. and then centrifuged at 1,000 rpm for 5 minutes to obtain supernatant and pellet fractions. The supernatant was discarded and the remaining pellets were collected, washed with PBS, and then centrifuged at 1,000 rpm for 5 minutes, and the supernatant was collected. The collected supernatant was filtered through a 100 μm mesh to remove tissue debris, followed by washing with PBS. The resulting cells were cultured in 10% FBS containing DMEM/F12 medium (WelGENE Inc.). After 24 hours of culture, non-adherent cells were removed by washing with PBS, and the adherent cells were cultured while the 10% FBS containing DMEM/F12 medium was replaced with fresh one at 2-day intervals, thereby obtaining human subcutaneous adipose tissue-derived stem cells.

### REFERENCE EXAMPLE 8

### Preparation of Polyacrylamide Gel

7.5% polyacrylamide gel was prepared using Mini-protean 3 Electrophoresis Set (Bio-rad). First, a casting frame was formed by fixing 1.0 mm glass plate to a frame. 4.94 ml of distilled water, 2.5 ml of 1.5 M Tris-HCl buffer, 2.5 ml of 30% acrylamide solution, 50 μL of 10% ammonium persulfate (APS) and 10 μL of TEMED (N,N,N′,N′-tetra methyl ethylene diamine) were added to a 50 -mL conical tube and sufficiently mixed. Then, 4.5 ml of the mixture was added to the 1.0 mm glass plate to prepare resolution gel. Then, 500 μL of distilled water was added thereto such that the gel was not dried. When the resolution gel was completely hardened, distilled water on the gel was removed. To prepare a stacking gel, 3.05 ml of distilled water, 1.25 ml of 0.5 M Tris-HCl buffer, 0.67 mL of 30% acrylamide solution, 25 μL of 10% APS and 5 μL of TEMED were added to a 50 mL conical tube and sufficiently mixed. Then, the mixture was poured onto the 1.0 mm glass plate, after which 15-well (20 μL) template was put therein, and then the mixture was hardened. Reagents used for the preparation of polyacrylamide gel were as follows:

1) 1.5 M Tris-HCl buffer: Tris base 18.17 g (Invitrogen), 20% SDS (Amersham Pharmacia Biotech) 2 mL, distilled water 80 mL, (pH 8.8)

2) 0.5 M Tris-HCl buffer: Tris base 6.06 g (Invitrogen), 20% SDS (Amersham Pharmacia Biotech) 2 mL, distilled water 80 mL, (pH 6.8)

3) 30% acrylamide solution: 29% acrylamide (Sigma), 1% bis-acrylamide (Sigma)

### EXAMPLE 1

### Preparation of Recombinant Expression Vector Expressing Recombinant Protein

### <1-1> Preparation of Recombinant Plasmid Having FGF2 Gene Cloned Therein

In order to prepare a plasmid having FGF2 gene cloned therein, the forward primer bFGF-F(EcoRI) having a nucleotide sequence of SEQ ID NO: 7 and a reverse primer FGF2-R(Hind) having a nucleotide sequence of SEQ ID NO: 8 were synthesized.

Using the primer pair, PCR was performed using as a template a whole gene extracted from human fibroblasts, thereby amplifying only fibroblast growth factor 2 (FGF2).

4 ng of the amplified FGF2 gene fragment, 50 ng of a pGEM-T vector and 1 μL of T4 DNA ligase were added to 5 μL of 2× ligation buffer included in pGEM-T vector system I (Promega). Then, distilled water was added to a final volume of 10 μL. The mixture was allowed to stand at room temperature for one hour, followed by reaction at 16° C. for 12 hours. After completion of the reaction, E. coli K12 TB1 was transformed with the ligated product, and a recombinant plasmid containing a target gene cloned therein was selected from the transformed bacterial cell and named “pGEM-FGF2”.

### <1-2> Preparation of Recombinant Expression Vector Containing MBP-FGF2 Recombinant Gene Fragment Cloned Therein

To fuse the linker maltose-binding protein (MBP) with FGF2 gene, the recombinant plasmid pGEM-FGF2 prepared in Example <1-1>was digested with the restriction enzyme EcoRI in Enzynomics buffers Ez buffer I, and then treated with the restriction enzyme HindIII in Enzynomics buffers Ez buffer II, after which the FGF2 fragment was isolated on agarose gel. The isolated FGF2 gene was treated with BAP to facilitate a subsequent ligation reaction. For BAP treatment, 7.5 μL of buffer (1 M Tris-HCl, pH 8.0, Bioneer) was mixed with 1 μL of BAP solution (Fermentas), after which VEGF gene was added thereto to a final volume of 50 μL, and then the mixture was allowed to react at 65° C. for 1 hour. The reaction product was electrophresed on agarose gel, and then irradiated by a UV transilluminator (Avegene) to cut the desired portion. Then, FGF2 gene fragment was isolated from the recovered gel fragment using gel extraction kit (Qiagene).

Meanwhile, the vector pMAL-c2X (New England Biolabs) having MBP gene for ligation was digested with EcoRI in Enzynomics buffers Ez buffer I, and then digested with HindIII in Enzynomics buffers Ez buffer II. Then, a MBP containing vector fragment was separated in an agarose gel.

9 μL of the FGF2 gene separated as described above, 3 μL of the digested vector fragment pMAL-c2X and 12 μL of enzyme solution I included in a DNA ligation kit (Ver 2.1, Takara) were mixed with each other. Distilled water was added thereto to make total volume of 20 μL, and then the mixture was subjected to ligation at 16° C. for 16 hours. After completion of the reaction, E. coli K12 TB1 was transformed with the ligated product, and a recombinant expression vector having a MBP-FGF2 fusion gene cloned therein was screened from the bacterial cells and named “pMAL-c2X-FGF2”. The transformed bacterial strain E. coli K12 TB1/pMAL-bFGF obtained by transforming E. coli K12 TB1 was deposited with Korean Collection for Type Cultures (KCTC), Korea Institute of Bioscience and Biotechnology, on Apr. 28, 2009 under accession number KCTC-11505BP.

The sequencing of the recombinant protein revealed that the amino terminus of fibroblast growth factor (FGF) is fused to the carboxyl terminus of maltose-binding protein (MBP), and the MBP-FGF recombinant protein capable of adhering to stem cells has an amino acid sequence of SEQ ID NO: 6 which is encoded by a polynucleotide having a nucleotide sequence of SEQ ID NO: 5.

### EXAMPLE 2

### Expression and Purification of MBP-FGF2 Recombinant Protein

### <2-1> Induction of expression of MBP-FGF2 Recombinant Protein

E. coli K12 TB1 was transformed with the recombinant expression vector pMAL-c2X-FGF2 expressing the MBP-FGF2 fusion protein, prepared in Example <1-2>, and was cultured at 37° C. in LB (Luria-Bertani) solid medium for one day. Next day, colonies formed on the medium were collected and inoculated in 3 mL of RB (rich medium+glucose) liquid medium containing 60 μg/mL ampicilline, followed by further culture at 37° C. for approximately 2 hours. IPTG (isopropyl-β-D-thiogalactopyranoside) was added thereto to a final concentration of 3 mM, followed by further culture at 37° C. for 2 hours. After completion of the culture, 1 mL of the culture solution was centrifuged to obtain cell pellets. 20 μL of 1× sample loading buffer was added to the cell pellets, which were then well mixed. The mixture was heated at 95° C. for 5 minutes and then cooled down to room temperature. Then, 15 μL of the mixture was subjected to electrophoresis on 10% SDS-polyacrylamide gel. After completion of the electrophoresis, the polyacrylamide gel was stained with Coomassie brilliant blue and analyzed by Western blotting using anti-MBP antiserum (New England Biolabs) to confirm whether the MBP-FGF2 fusion protein was expressed.

### <2-2> Expression and Purification of MBP-FGF2 Recombinant Protein

E. coli cells transformed with the recombinant plasmid pMAL-c2X-FGF2 in Example <2-1>were inoculated in RB medium containing 60 μg/mL amplicillin, followed by culture for overnight at 37° C. 10 mL of the culture solution was added to 1 liter of RB medium, followed by shaking-culture at 37° C. When the OD₆₅₀ of the culture solution reached approximately 0.6, IPTG was added to a final concentration of 3 mM. 2 hours after the addition of IPTG, culture was stopped. The culture solution was centrifuged (Combi-514R, Hanil) at 4000×g for 20 minutes to collect cell pellets. The cell pellets were resuspended in 50 mL of buffer (1 M Tris-HCl 20 mL, pH 7.5, NaCl 11.7 g, 0.5 M EDTA 2 mL), to which EDTA (ethylenediaminetetraacetic acid) and PMSF (phenylmethanesulphonyl fluoride) were then added to a final concentration of 1 mM. The cell culture mixture was frozen (−20° C.) and thawed repeatedly before purification in order to facilitate cell lysis. The cells were lysed using a sonicator (Fisher Scientific Model 500 Sonic Dismembrator) with a 10% output for approximately 10 seconds on ice bath. Then, the cell lysate was allowed to stand on ice bath for 30 seconds. The above procedure was repeated twice for complete cell lysis. The cell homogenate thus obtained was centrifuged (Combi-514R, Hanil) at 9000×g for one hour to collect the supernatant containing water-soluble protein, which was then 5-fold diluted with buffer (1 M Tris-HCl (20 mL), pH 7.5, NaCl (11.7 g), 0.5 M EDTA (2 mL)).

To separate the MBP-FGF2 recombinant protein expressed in the E. coli transformant, affinity chromatography was performed using amylase resin (New England Biolabs). This column was equilibrated by washing with 8× bed volume buffer (1 M Tris-HCl (20 mL), pH 7.5, NaCl (11.7 g), 0.5 M EDTA (2 mL)). The supernatant containing soluble protein obtained above was loaded into the equilibrated amylase resin affinity chromatography at a speed of 1 mL per minute. Non-adsorbed proteins were removed by running with 12× bed volume buffer (1 M Tris-HCl (20 mL), pH 7.5, NaCl (11.7 g), 0.5 M EDTA (2 mL)). The proteins adsorbed on the resin was eluted by adding 10 mM maltose elution buffer (1 M Tris-HCl (20 mL), pH 7.5, NaCl (11.7 g), 0.5 M EDTA (2 mL), 10 mM maltose) at a speed of 1 mL per minute. The recovered protein was subjected to electrophoresis (Bio-rad) on 10% polyacrylamide gel to examine the molecular weight and purity of the purified protein. As a result, the purified protein had a purity of at least 95% and a molecular weight of about 60,000 Da.

The protein sample was placed in a dialysis membrane (MWCO12-14,000, Spectrum laboratories, Inc.) and dialyzed with PBS for 3 days, thus obtaining a protein from which maltose has been removed. Then, the protein was concentrated by centrifugation (Combi-514R, Hanil) at 4000×g for 45 minutes using Centrifugal Filter (Amicon Ultra-15 MWCO 5,000, Millipore). The concentrated protein was named “MBP-FGF2”.

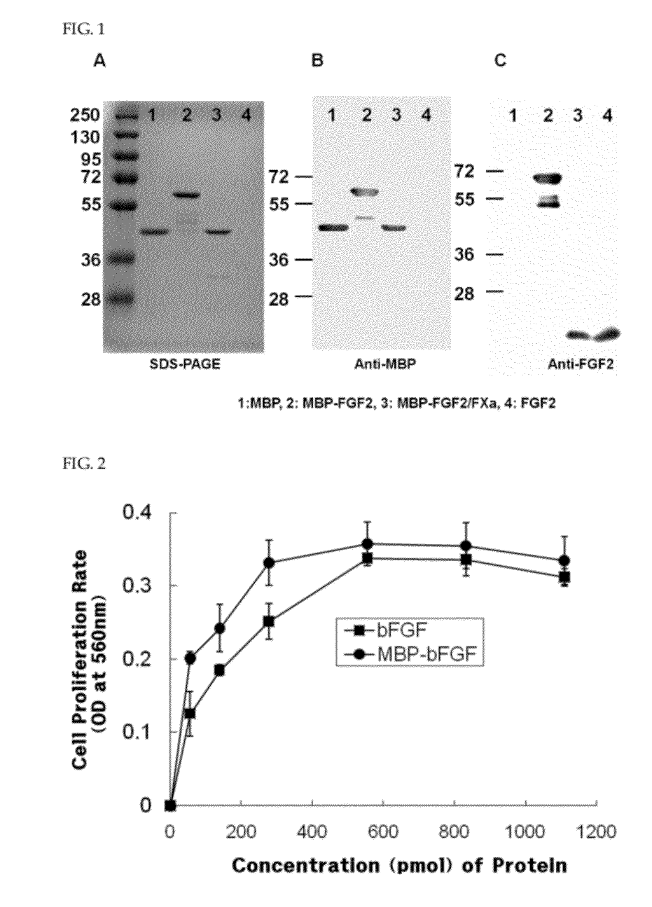

FIG. 1 shows the results of SDS-PAGE analysis of the MBP-FGF2 recombinant protein, separated and purified as described above. In FIG. 1, “A” shows the results of Coomassie blue staining of the SDS-PAGE gel, “B” shows the results of Western blot analysis of the gel, conducted using anti-MBP, and “C” shows the results of Western blot analysis of the gel, conducted using anti-FGF. In FIGS. 1A, 1B and 1C, lane 1: MBP alone; lane 2: MBP-FGF2 fusion protein; lane 3: MBP-FGF2 fusion protein treated with FXa; and lane 4: FGF2 alone.

As can be seen in FIG. 1, the MBP-FGF2 recombinant protein had a higher molecular weight than MBP and FGF2 alone. Also, 1 μg of FXa was added and allowed to react with 20 μg of the MBP-FGF2 recombinant protein at room temperature for 4 hours, the recombinant protein was separated into MBP and FGF2. This suggests that the MBL-FGF2 recombinant protein expressed in the E. coli transformant was successfully separated and purified from the transformant.

### EXAMPLE 3

### Measurement of Activity of MBP-FGF2 Recombinant Protein

To measure the activity of the MBP-FGF2 fusion protein, the following test was performed on human umbilical vein endothelial cells (HUVEC; Modern Cell & Tissue Technologies). HUVEC cells express the FGF2 receptor FGFR1 and are used to measure the activity of FGF2, etc. HUVEC cells were suspended in endothelial growth medium-2 (EGM-2, Lonza) and seeded in a 96-well plate at a cell density of 2×10³/well. 4 hours after cell seeding, the medium was replaced with FGF2-free EGM-2 (basal medium) or with EGM-2 media containing FGF2 (R&D Systems) or the inventive MBP-FGF2 recombinant protein at a concentration of 0, 55, 138, 277, 555, 832 or 1100 pmol in order to stimulate the HUVEC cells, followed by culture for 3 days. After completion of culture, the number of proliferated cells was counted with a cell counting kit (Dojindo Laboratories).

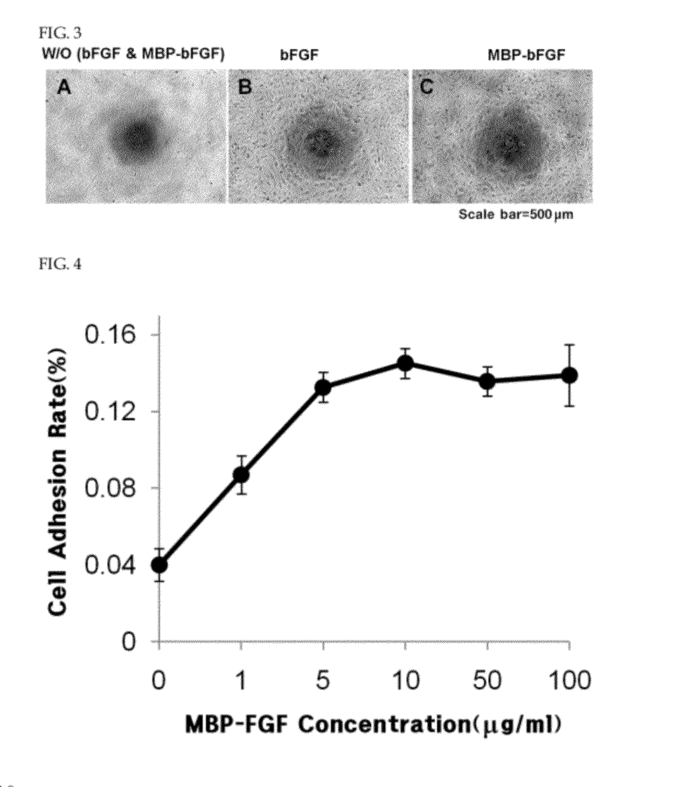

FIG. 2 is a set of graphs showing the number of proliferated cells as a function of the concentration of the protein, 3 days after treatment. As can be seen therein, the number of proliferated cells increased as the treatment concentrations of FGF2 and MBP-FGF2 increased. FIG. 3 shows a set of cells cultured for 3 days in media containing 1100 pmol of each of FGF2 and MBP-FGF2. As can be seen therein, when cells were cultured in basal medium, they did not substantially proliferate, whereas cells were cultured in medium containing MBP-FGF2, the cells did proliferate to an extent similar to that observed when FGF2 was added. Such results suggest that, even if FGF2 is expressed in the form of a recombinant protein with MBP and purified, it maintains its original activity.

### EXAMPLE 4

### Measurement of Activity of Adhesion of Adipose-Derived Stem Cells to Surface Immobilized with MBP-FGF2 Recombinant Protein

In order to examine the activity of adhesion of adipose-derived stem cells to a surface immobilized with the MBP-FGF2 recombinant protein, the following test was carried out. In a clean bench (Sanyo), the MBP-FGF2 recombinant protein, separated and purified in Example 2 above, was filtered through a 0.22 μm syringe filter (Millex GV, Millipore). Then, 100 μL of the protein was added to 96-well plates (non-tissue culture, Falcon) at varying concentrations of 1, 5, 10, 50 and 100 μg/mL and allowed to stand for 4 hours in the clean bench such that it was immobilized on the plate surface. Then, the 96-well plates were washed three times with 200 μL of PBS. Also, in order to prevent non-specific binding between the plate immobilized with the MBP-FGF2 recombinant protein and the adipose-derived stem cells, 200 μL of 1% BSA (bovine serum albumin, Sigma) was added to some wells of the plates which were then incubated in a clean bench for 2 hours and washed three times with 200 μL of PBS.

Adipose-derived stem cells were seeded in 96-well plates immobilized with the MBP-FGF2 recombinant protein at a cell density of 8×10³ cells/well. Herein, serum-free DMEM/F12 medium (WelGENE Inc.) was used. Then, the cells were cultured in an incubator (Thermo) at 37° C. for 1 hour, after which the amount of adherent adipose-derived stem cells was measured by a BCA (bicinchoninic acid) protein assay, thereby quantifying the degree of adhesion of cells.

FIG. 4 shows the results of measuring the rate of adhesion of adipose-derived stem cells to the polystyrene surface immobilized with the MBP-FGF2 recombinant protein of the present invention. As can be seen therein, the adipose-derived stem cells showed the highest adhesion rate on the surface immobilized with 10 μg/mL or more of the recombinant protein.

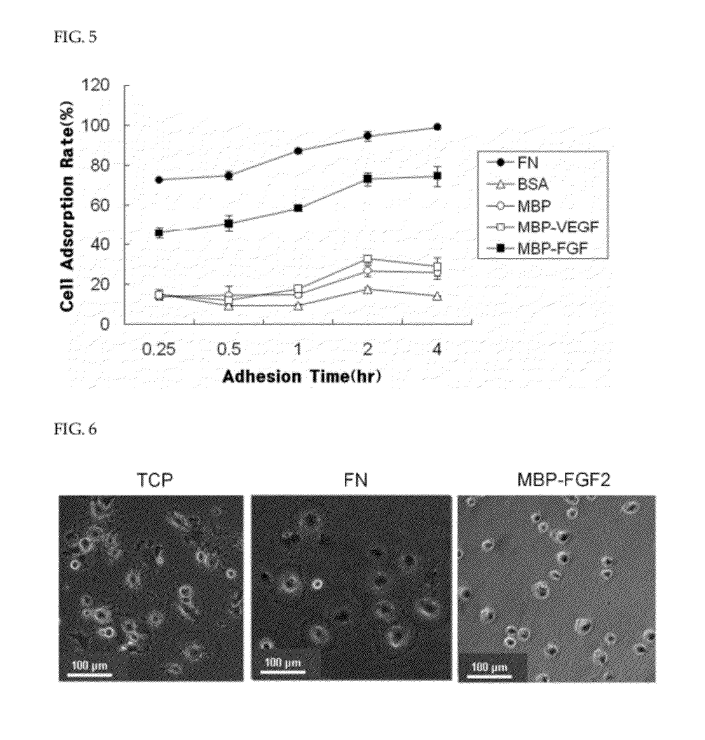

FIG. 5 shows the results of measuring the rates of adhesion of adipose-derived stem cells to various culture surfaces as a function of culture time. As can be seen therein, on the surfaces immobilized with each of MBP and the MBP-VEGF recombinant protein, the rate of adhesion of adipose-derived stem cells was as low as 30%, even 4 hours after cell seeding. On the surface immobilized with the MBP-FGF2 recombinant protein according to the present invention, the adhesion rate of cells was about 60% of the cell adhesion rate on the fibronectin (FN)-immobilized surface in the initial stage of culture, but it increased to about 80% after 4 hours of culture. Such results suggest that the MBP-FGF2 recombinant protein according to the present invention has an activity of adhering specifically to adipose-derived stem cells.

FIG. 6 is a set of phase-contrast micrographs showing the results of observation with a phase-contrast microscope (Nikon) for a change in morphology of adipose-derived stem cells cultured on the hydrophobic polystyrene surface immobilized with the MBP-FGF2 recombinant protein of the present invention in the absence of serum. As can be seen therein, when adipose-derived stem cells were cultured on commercial tissue cell tissue plate (TCP), pseudopodia were formed around circular cells. When adipose-derived stem cells were cultured on the surface immobilized with fibronectin (FN), a natural ECM component, the cells were perfectly spread, and when adipose-derived stem cells were cultured on the surface immobilized with the MBP-FGF2 recombinant protein, the morphology of the cells was maintained at a circular shape. Such results indicate that immobilization of the inventive MBP-FGF2 recombinant protein on a culture plate surface plays an important role in proliferating adipose-derived stem cells while maintaining the morphology of the cells.

### EXAMPLE 5

### Activity of Adhesion of Bone Marrow-Derived Mesenchymal Stem Cells to Surface Immobilized with MBP-FGF2 Recombinant Protein

In order to investigate the activity of adhesion of human bone marrow-derived mesenchymal stem cells (obtained from the Stem Cell Therapy Center, the Yonsei University College of Medicine) to a surface immobilized with the MBP-FGF2 recombinant protein, 96-well plates, the surface of which was immobilized with the MBP-FGF2 recombinant protein as described in Example 4 above, were prepared.

Bone marrow-derived stem cells were seeded in 96-well plates immobilized with the MBP-FGF2 recombinant protein at a cell density of 1×10⁴ cells/well. Herein, serum-free high-glucose DMEM medium (WelGENE Inc.) was used. The seeded cells were cultured in an incubator (Thermo) at 37° C. for 1 hours, after which the amount of adherent bone morrow-derived mesenchymal stem cells was measured by a BCA (bicinchoninic acid) protein assay, thereby quantifying the degree of adhesion of cells.

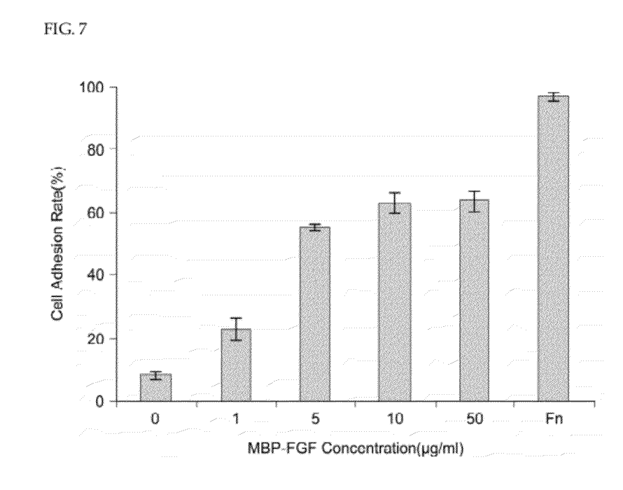

FIG. 7 shows the results of the rate of adhesion of bone marrow-derived mesenchymal stem cells to the polystyrene surface immobilized with the MBP-EGF2 recombinant protein of the present invention as a function of the concentration of the protein. As can be seen therein, the bone marrow-derived mesenchymal stem cells showed the highest adhesion rate at the surface immobilized with 10 μg/mL or more of the recombinant protein, in a manner similar to Example 4. Herein, the rate of adhesion of the bone marrow-derived mesenchymal stem cells to the surface immobilized with the MBP-EGF2 recombinant protein of the present invention was about 60% of the rate of cell adhesion to the fibronectin (Fn)-immobilized surface and was similar to the rate of adhesion of adipose-derived stem cells in Example 4. Such results suggest that the MBP-FGF2 recombinant protein according to the present invention has an activity of adhering specifically to bone marrow-derived mesenchymal stem cells.

Although several exemplary embodiments of the present invention have been described for illustrative purposes, those skilled in the art will appreciate that various modifications, additions and substitutions are possible, without departing from the scope and spirit of the invention as disclosed in the accompanying claims.

1. A method of culturing stem cells using immobilized fibroblast growth factor (FGF), the method comprising the steps of:
1) preparing a recombinant protein of FGF-polypeptide linker capable of adhering to stem cells, in which a polypeptide linker is fused to the amino terminus of FGF;
2) immobilizing the recombinant protein on a culture plate having a hydrophobic surface using the amino terminus of the polypeptide linker; and
3) allowing stem cells to bind to the recombinant protein-immobilized culture plate and culturing the stem cells.

2. The method of claim 1, wherein the recombinant protein in step 1) is an MBP-FGF recombinant protein in which maltose binding protein (MBP) as a polypeptide linker is fused to the amino terminus of FGF.

3. The method of claim 2, wherein the MBP-FGF recombinant protein has an amino acid sequence set forth in SEQ ID NO: 6.

4. The method of claim 2, wherein step 2) is carried out by immobilizing 5-100 μg/mL of the MBP-FGF recombinant protein on the hydrophobic surface.

5. The method of claim 1, wherein the hydrophobic surface in step 1) is a silanized surface, a hydrocarbon-coated surface, a polymer surface or a metal surface.

6. The method of claim 5, wherein the polymer is selected from the group consisting of polystyrene, polycarbonate, polypropylene, polyethylene, Teflon, polytetrafluoroethylene, and polyester-containing biodegradable polymers.

7. The method of claim 5, wherein the metal is selected from the group consisting of stainless steel, titanium, gold and platinum.

8. The method of claim 1, wherein the recombinant protein in step 2 is immobilized on the hydrophobic surface by spontaneous physical adsorption.

9. The method of claim 8, wherein the physical adsorption is achieved by allowing the recombinant protein and the hydrophobic surface to react at 4˜25° C. for 1-24 hours.

10. The method of claim 1, wherein the FGF immobilized on the hydrophobic surface in the form of a recombinant protein in step 2 is exposed to the outside.

11. The method of claim 1, wherein the FGF immobilized on the hydrophobic surface in the form of a recombinant protein in step 1 maintains 50% or more of its physical activity or function.

12. The method of claim 1, wherein the stem cells in step 3) are selected from the group consisting of adipose-derived stem cells, mesenchymal stem cells, bone marrow stem cells, umbilical cord blood stem cells, and neural stem cells.

13. An MBP-FGF recombinant protein capable of adhering to stem cells, in which the carboxyl terminus of maltose-binding protein (MBP) is fused to the amino terminus of fibroblast growth factor.

14. A polynucleotide encoding the MBP-FGF recombinant protein of claim 13.

15. The polynucleotide of claim 14, wherein he polynucleotide has a nucleotide sequence set forth in SEQ ID NO: 5.

16. A recombinant expression vector comprising the polynucleotide of claim 14.

17. The recombinant expression vector of claim 16, wherein the recombinant expression vector is pMAL-c2X-FGF2.

18. A bacterial strain transformed with the recombinant expression vector of claim 16.

19. The bacterial strain of claim 18, wherein the bacterial strain is E. coli K12 TB1/pMAL-bFGF (accession number: KCTC-11505BP).

20. A method for producing the MBP-FGF recombinant protein of claim 11, the method comprising a step of culturing the bacterial strain of claim 18 and then recovering the recombinant protein from the culture.

21. The method of claim 20, wherein the recombinant protein is isolated and purified from the culture using maltose-specific affinity column chromatography.
